(19) United States
(12) Patent Application Publication
(10) Pub. No.: US 2003/0216765 A1
(43) Pub. Date: Nov. 20, 2003

(54) Device for cutting cartilage to size

(76) Inventor: Andreas Efinger, Hinterm Hofle (DE)

Correspondence Address: ST. ONGE STEWARD JOHNSTON & REENS, LLC, 986 BEDFORD STREET, STAMFORD, CT, 06905-5619, US

(21) Appl. No.: 10/430,069

(22) Filed: May 6, 2003

(30) Foreign Application Priority Data
May 6, 2002 (EP) 02 010 047.5

(51) Int. Cl. A61B 17/32

(52) U.S. Cl. 606/167

(57) ABSTRACT

A device for cutting cartilage to size has a first body and a second body between which the cartilage to be cut to size can be placed. It is proposed that the second body has at least one opening extending through it, which opening serves as a seat for the cartilage, and the perichondrium surrounding the cartilage can be held between the bodies.

This application claims priority from pending European Patent Application No. 02010047.5 filed May 6, 2002.

### BACKGROUND OF THE INVENTION

The invention relates to a device for cutting cartilage to size, with a first body and a second body between which the cartilage to be cut to size can be placed, and with a seat in which the cartilage can be received.

A device of this kind is known from DE 40 34 615 A1.

Such medical cutting devices are used to cut a cartilage to a specific size.

In some surgical interventions, thin slices of cartilage are needed, for example for plastic correction of an eardrum defect. The pieces of cartilage which are taken from other organs, for example from the nasal septum, are too thick for this purpose and therefore have to be cut to a desired size and shape.

Preparation by hand using pincers and scissors or a scalpel is extremely difficult, so that use is made of medical cutting devices of this kind.

In the above-mentioned device, a seat is cut out in the first body and is open towards a front face of said body. The second body which can be placed on the first one closes this seat like a lid. The cartilage is introduced into the space which is thus created and open towards the front face.

A guide slit is provided at a predetermined distance parallel to the base surface of the seat, which guide slit serves as a guide for a cutting blade, for example in the form of a razor blade. In this way, the cartilage held in the seat between the first body and second body can be divided into two parts, that is to say cut to size, from the outside by insertion of the cutting blade. By inserting precisely dimensioned spacer leaves into the recess, correspondingly thinner slices of cartilage can be obtained.

Particularly with regard to variability, this device has disadvantages since, in order to change the desired cartilage thickness, very fine spacer leaves have to be introduced into what is already a very small seat anyway. It should be borne in mind that, for example for correcting eardrum defects, these implants have a size in the range of millimetres.

If different sizes and different shapes of cartilage are now wanted depending on the particular anatomy of the patient, then differently configured devices with the correspondingly numerous spacer leaves have to be kept in stock.

In the repair of eardrum defects, it is desirable for a certain amount of the perichondrium to be retained around the cartilage.

This serves to connect the graft via the perichondrium to the actual eardrum, from which the cut cartilage protrudes, and serves as a support for the hammer, or auditory bone, via which the vibrations of the eardrum are taken up following repair.

In the above-mentioned device it is not possible, nor is provision made, for the perichondrium around the cartilage to be left in place on the cartilage.

It is, therefore, an object of the present invention to improve a device of the above-mentioned type in such a way that a high degree of flexibility in respect of size and shape of the cartilage to be prepared is achieved using robust and easy-to-handle components, and in such a way that it is also possible, during preparation, for the perichondrium surrounding the cartilage to be left in place on the latter.

### SUMMARY OF THE INVENTION

According to the invention, this object is achieved by the fact that the second body has at least one opening extending through it, which opening serves as a seat for the cartilage, and the perichondrium surrounding the cartilage can be held between the bodies.

The seat is thus no longer designed as a seat cut out in the first body, but as a through-opening cut out in the second body. In this way it is possible for the perichondrium surrounding the cartilage to be placed flat between the first body and the second body and to be held between these two bodies. The cartilage to be prepared extends through the opening in the second body and is cut to size by means of the portion protruding through this opening of the second body being cut off. Since the perichondrium surrounding the cartilage is held between the first and second bodies, it cannot come into contact with the cutting tool, so that the preparation can be done without damaging the perichondrium.

Through the choice of shape, size and thickness of the second body, it is then possible to adapt it to the desired cartilage size.

For this purpose, it is advantageous if the second body has a plurality of openings of different size and/or different shape.

The openings can be circular, oval, or of any other desired shape.

For this purpose, it is particularly advantageous if the second body is designed as a plate-shaped stencil.

This measure has the advantage that the stencil can simply be placed on the first body, and the cartilage to be prepared, that is to say cut to size, protrudes through its openings. The perichondrium is fixed flat between the two bodies and safe from the cutting tool.

The variation in the thickness of the cartilage is achieved quite simply by providing a plurality of second bodies of different thickness.

It is also possible to realize all of these parameters in a single plate or stencil depending on how large the device is configured, that is to say a stencil with differently shaped holes, with different sizes of holes, and with areas of different thickness.

In a further embodiment of the invention, the first body is designed as a block on whose flat upper face the second body can be placed.

This measure has the advantage that the physician carrying out the preparation work has an easy-to-handle device which also resists the cutting pressure during cutting. In the actual preparation work, the piece of cartilage not yet cut to size and the perichondrium around it are placed on the flat upper face of the block, the flat stencil is then placed on top, and then, by a simple cutting procedure in which the cutting blades are guided across the upper second body, the cartilage is correspondingly cut off, that is to say the piece which protrudes through and extends above the opening of the second body.

In a further embodiment of the invention, the first body and second body can be placed one on top of the other and in such a way as to be fixed in position relative to one another. This measure has the advantage that, in the actual cutting procedure in which a certain force is exerted, the two bodies do not move relative to one another.

The positionally fixed arrangement can be achieved by various measures such as clamps, catches or the like, and in a very simple configuration at least two pins project from the first body, via which pins the second body which has corresponding through-bores can be attached.

In a further embodiment of the invention, suction openings are cut out in the first body and open out towards the face on which the second body can be placed.

This measure has the advantage that a graft placed on the upper face of the first body can be drawn by suction via the suction openings. For this purpose, the suction openings are connected to a suitable underpressure source.

In a further embodiment of the invention, a holding-down tool is provided by means of which a cartilage located between the first body and second body and protruding through an opening in the latter can be held down.

This measure has the advantage that, in the actual cutting procedure, the cartilage, that is to say the piece protruding above the opening in the second body, is held down. By this means, it is possible to rule out the possibility of the cartilage being pulled out of the opening by the cutting tool.

In a further embodiment of the invention, a cutting tool is provided by means of which a portion of cartilage protruding through the opening in the second body can be cut off.

This measure has the advantage that the geometry of the cutting tool can be optimally adapted to the corresponding surface of the second body, so that the cartilage can be suitably prepared with one cut.

It will be appreciated that the aforementioned features, and the features still to be explained below, can be used not only in the stated combinations, but also in other combinations or in isolation, without departing from the scope of the present invention.

### BRIEF DESCRIPTION OF THE DRAWINGS

The invention is described and explained in more detail below on the basis of a number of chosen illustrative embodiments and with reference to the attached drawings, in which:

FIG. 1 shows a perspective exploded view of a first illustrative embodiment of a device according to the invention,

FIG. 2 shows a cross section along the line II-II in FIG. 1 in the assembled state of the device upon preparation of a cartilage,

FIG. 3 shows a plan view of an upper face of a first body in a second illustrative embodiment of a device according to the invention, with a suction device, and

FIG. 4 shows a cross section along the line IV-IV in FIG. 3.

### DETAILED DESCRIPTION OF PREFERRED EMBODIMENTS

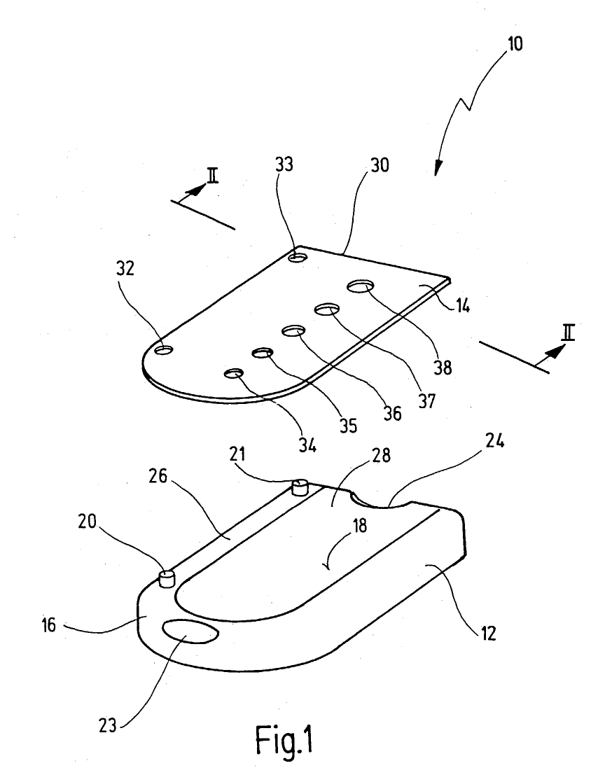

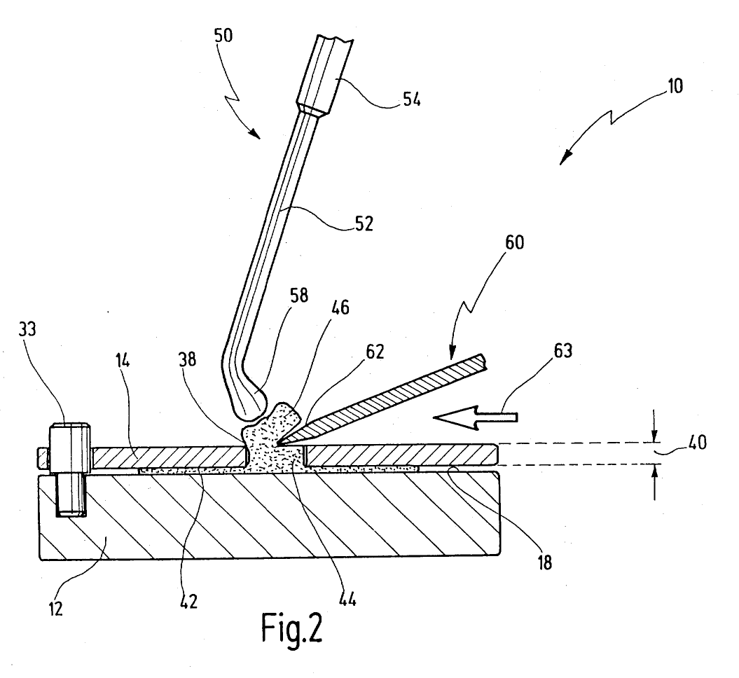

In FIGS. 1 and 2, a device for cutting a cartilage 44 to size is designated overall by reference number 10.

The device 10 has a first body 12 and a second body 14.

The first body 12 consists of a solid block 16 onto whose upper face 18 the second body 14 can be placed.

Protruding from the upper face 18 of the block 16, at its edge, there are two pins 20 and 21 which serve to positionally fix a second body 14 placed thereon, as will be described below.

At opposite ends, finger depressions 23 and 24 are cut out in the upper face and serve to make it easier to lift off a second body 14, placed on the upper face 18 after the manual operation.

The block 16 is made up of a U-shaped frame 26 in which a cutting plate 28 is fixed.

The second body 14 is designed as a plate-shaped stencil 30 which at its side has two through-bores 32 and 33 via which the stencil 30 can be fitted onto the two pins 20 and 21.

The stencil 30 is provided with a row of through-openings 34 to 38, which are circular in the illustrative embodiment shown and which have different diameters.

The diameters in this case vary from 6 to 10 mm.

FIG. 2 shows a situation in which a portion of a perichondrium 42 is first placed on the upper face 18 of the block 16, a cartilage 44 protruding upwards from one side of this. The cartilage 44 is now so positioned that, as is shown in FIG. 2, it protrudes through the opening 38 when the plate-shaped stencil 30 is placed onto the block 16. The cartilage 44 has such a height that a portion 46 protrudes above the upper face of the plate-shaped stencil 30.

With the device, the cartilage 44 is now to be cut to a height corresponding to the thickness 40 of the stencil 30. If the height of the cartilage 44 is to be smaller or greater, correspondingly thinner or thicker stencils 30 are fitted.

It can also be seen from the cross-sectional view in FIG. 2 that the perichondrium 42 around the cartilage 44 comes to lie flat between the upper face 18 of the block 16 and the underside of the stencil 30.

In the actual cutting procedure, a cutting tool 60 is guided across the upper face of the stencil 30, as is indicated by an arrow 63 in FIG. 2, its cutting edge 62 cutting off the portion 46 of the cartilage 44 protruding over the stencil 30.

To ensure that the cartilage 44 is not pulled out from the opening 38 by the cutting tool 60, a holding-down tool 50 is also provided.

The holding-down tool 50 has an elongate shaft 52 which at one end is provided with a grip 54 and at the other end is provided with an angled ram 56.

The ram 56 is designed in such a way that it can be placed on the cartilage 44 and holds this down.

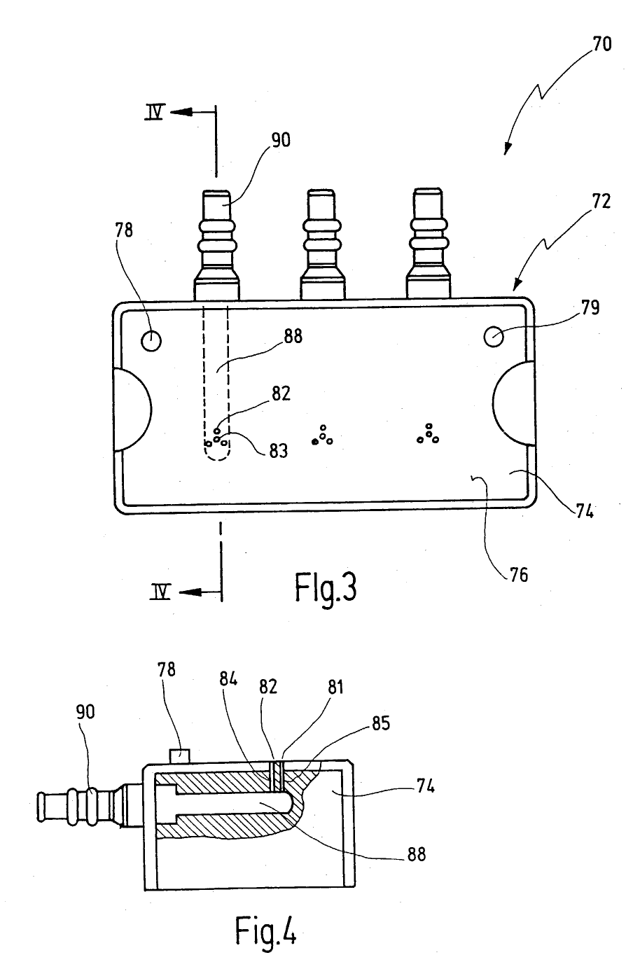

FIGS. 3 and 4 show a further illustrative embodiment of a device according to the invention which is indicated in general by reference number 70.

In FIGS. 3 and 4, only the first body 72 is shown. In this case too, a second body is also provided which is designed like the first one, that is to say a plate-shaped stencil with correspondingly shaped openings.

As can be seen from the plan view in FIG. 3, a plurality of suction openings open out on the upper face 76 of the first body 72 likewise designed as a block 74. Only two suction openings 82 and 83 are labeled here. The two pins 78 and 79 once again protrude from the upper face 76 so that the stencil to be placed on top is fixed in the correct position.

From the cross-sectional view in FIG. 4, it will be seen that the suction openings 82 and 83 communicate via channels 84 and 85 with a bore 88, which in turn communicates with a lateral suction attachment 90. Via this attachment 90, the block 74 can be connected to an underpressure suction device. When a cartilage with surrounding perichondrium is placed in the area of the suction openings 82 and 83, this piece is thus held by the applied underpressure.

From the view in FIG. 3 it will be seen that a plurality of such suction openings and corresponding bore attachments are provided which are so positioned that they lie under the openings in the plate-shaped stencil (not shown here).

In this case, as a result of the fixing by means of the underpressure, a holding-down tool may no longer be necessary. However, for reasons of safety, or for other reasons, a holding-down means 50 may still quite conceivably be used in the cutting procedure.

What is claimed is:

1. A device for cutting a cartilage to size, having
a first body, and
a second body
between which first and second body a cartilage to be cut to size can be placed,
wherein said second body having at least one opening extending through it, which opening serves as a seat for said cartilage, a perichondrium surrounding said cartilage can be held between said first and said second body.

2. The device of claim 1, wherein said second body has a plurality of openings of different sizes.

3. The device of claim 1, wherein said second body has a plurality of openings of different shapes.

4. The device of claim 1, wherein said second body being designed as a plate-shaped stencil.

5. The device of claim 1, wherein a plurality of second bodies of different thickness are provided.

6. The device of claim 1, wherein said first body is designed as a block, having a flat upper surface, onto which upper surface said second body can be placed.

7. The device of claim 5, wherein said first body and said second body can be placed one on top of the other in such a way as to be fixed in a position relative to one another.

8. The device of claim 1, wherein suction openings are cut out in said first body, said openings open towards a face onto which said second body can be placed.

9. The device of claim 1, wherein a holding-down tool is provided by means of which tool a cartilage located between said first body and said second body and protruding through an opening in said second body can be held down.

10. The device of claim 1, wherein a cutting tool is provided by means of which a portion of a cartilage protruding through an opening in said second body can be cut off.
